(19) United States
(12) Patent Application Publication
(10) Pub. No.: US 2007/0244373 A1
(43) Pub. Date: Oct. 18, 2007

(54) Measurement Device

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/578,135

(22) PCT Filed: Apr. 6, 2005
(86) PCT No.: PCT/EP05/03594

§ 371 Date: May 2, 2006

(30) Foreign Application Priority Data
May 13, 2004 (DE) 10 2004 023 527.9

(51) Int. Cl. A61B 5/00

(52) U.S. Cl. 600/301

(57) ABSTRACT

A measurement device for sensing medical parameters in a human body can be accommodated in a body cavity, in particular in a blood vessel, and has at least one sensor and one holder. In order to temporarily or permanently implant and subsequently remove the measurement device from the body cavity in a simple manner, the holder has at least one first and one second magnetic element, at least one of the magnetic elements being a magnet, with one of the elements being arranged inside and the other outside the body cavity, and the measurement device can be fixed in the body cavity by the holder.

### BACKGROUND

The invention relates to a measurement device for sensing medical parameters. This device can be accommodated in a body cavity, in particular a blood vessel, and has at least one sensor and one holder.

In the course of advancing miniaturization in measurement technology, sensors that obtain measurement values of relevant parameters directly in the human body and that can analyze these values immediately if necessary are gaining more and more importance under the headings of micro-technology and nanotechnology. In addition to other applications, the main application of such sensors lies in the analysis of blood values or blood vessels (measurements for glucose, blood pressure, temperature, pH, general blood analysis, etc.), which means that the sensor must have direct contact with the medium to be analyzed. In addition, it can be desirable, for example, for testing vessels frequently found at the surface of the body, to couple electromagnetic radiation, for example, in the form of visible light, in the region of a sensor concerning a corresponding evaluation.

Here, there is the problem that available sensors can be introduced into the corresponding vessels, but can be anchored there only with difficulty, especially when dealing with blood vessels, without hindering or significantly affecting the blood flow. Another problem is that after successful measurement or a series of measurements, the sensor frequently should be removed again.

### SUMMARY

Therefore, there is the objective of making available a measurement device, which can be fixed within vessels temporarily or permanently and can be easily removed again if necessary.

This objective is met for a measurement device of the above-noted type, in that the holder has at least one first and one second magnetic element, of which at least one is a magnet, and of which one is arranged inside the body cavity and one is arranged outside the body cavity, and that the measurement device can be fixed in the body cavity by the holder. By utilizing the magnetic force generated between the magnetic elements, the measurement device can be fixed to the vessel wall in a simple way. Here, the magnetic force penetrates through the vessel wall and exerts an attractive force between the approximately opposing magnetic elements on both sides of the vessel wall. This attractive force is sufficiently strong to hold the measurement device to the vessel wall against its own weight and a possible flow of medium through the body cavity. If necessary, this fixation can be disabled again simply by removing the magnetic element on the outer vessel wall and then the measurement device can be removed.

For preferred improvements of the measurement device according to the invention, either one of the two magnetic elements is a magnet and the other is a part made from a ferromagnetic material or both magnetic elements are embodied as magnets.

To keep the measurement device to be inserted into the vessel as small and compact as possible and thus, e.g., to cause only minimal interference of the blood flow in a blood vessel, it is advantageous in one embodiment of the measurement device if the sensor is connected rigidly to the magnetic element arranged inside the body cavity.

Preferably, in another embodiment of the measurement device, the magnetic element arranged outside the body cavity is the magnet. From the outside wall of the vessel, this magnetic element is in the position to fix the sensor provided with the other magnetic element. Here, this outer magnet can be applied, for example, both to the surface of the body and also especially initially implanted and fixed subcutaneously itself, wherein the sensor is implanted with the magnetic element and can be fixed by the already provided magnets. For the fixation of the sensor, other holding means, such as, for example, a T-bar mounted especially in the downstream direction, can also be used.

For multiple use of one or more sensors for measuring values at different locations in the body cavity or in several connected body cavities, for example, along the course of a vessel and/or for the best possible placement of the measurement device, it is advantageous if the measurement device can be moved by rearranging or shifting the magnets. The sensor with the magnetic element can be shifted along with the movement of the magnet along the outer vessel wall in a simple way and then fixed at a new location. Simultaneously, it can be tested whether the sensor has already become ingrown during its dwell time in the body cavity and therefore it is either to be removed or the magnet can be done without if a longer dwell time is desired in the future.

If several different or also the same measurement values are to be detected simultaneously due to a wide range of reasons, for example, due to time constraints, it is advantageous for an improvement of the measurement device if there are several sensors, which are provided with magnetic elements and which can be fixed by at least one magnet in the body cavity. Here, very different arrangements of sensors in the body cavity, for example in a line or in a plane, can be imagined.

Preferably, the measurement device according to the invention can be inserted into the body cavity or mounted to its outer wall both in terms of the sensors with the magnetic elements and also in terms of the magnets by means of an implantation instrument, a catheter, or the like. The magnetic element arranged outside the body cavity can also be applied especially preferably to the surface of the body itself or subcutaneously.

So that the elements of the measurement device to be inserted into the body do not lead to undesired rejection reactions, special requirements are placed on the compatibility. Advantageously, the measurement device can be at least partially sheathed or encased in a flexible, biocompatible material, especially in silicone. In this way, the measurement device can also be adapted particularly well to the corresponding vessel wall.

For protecting the measurement device from damage due to physiological fluids, an improvement of this device and especially of the electronic components arranged in this device are provided with an additional coating. This coating can also contribute to increased compatibility of the foreign body located in the body cavity.

For automatic operation of the measurement device according to the invention over a longer time in measurement and/or evaluation operation, this device is preferably provided with a power supply, especially a battery or an accumulator.

In a similar way, it is beneficial for a long-term independent operation of the measurement device if this device is provided in another embodiment in the region of the sensor or one of the magnetic elements with an electronic memory unit for temporary storage of the data detected by the sensors, so that the detected values can be fed from this memory unit to an external memory or evaluation unit at more or less regular intervals. In a preferred improvement, the measurement device can also be provided with its own evaluation unit in the region of the sensor or one of the magnetic elements, so that when the data already processed in this evaluation unit is queried, the prepared results of the corresponding measurements are already ready, which then permits quick analysis and diagnosis.

In preferred improvements of the measurement device, the sensors are those for detecting values of pressure, the blood-sugar level, the hemoglobin count, the oxygen and carbon dioxide partial pressures and content, and/or other relevant values of the body cavity and/or the medium located therein.

Because it is frequently desirable, in addition to a measurement device, to also provide the ability to introduce materials into a body cavity, the measurement device according to the invention is provided in one embodiment with at least one storage device for storing a material, especially a medicine, to be introduced into the body cavity. The contents of the storage device can then be emptied at a desired time into the body cavity. Here, naturally several storage devices can also be used simultaneously and one or more sensors can be allocated to these devices for their use. In one advantageous improvement, a dosing element for controlled release of the material is provided on the storage device, so that this can release, for example, uniform doses at regular intervals. Especially preferred is an improvement of the measurement device, in which this device is part of a control loop and the dosing element releases the material as a reaction to a measurement value detected by a sensor, so that, e.g., in an emergency, only a small amount of time elapses until the administration of a counteracting medicine.

For further processing and evaluation of the data of the measurement values detected by the sensors and possibly temporarily stored or even already evaluated, in another embodiment of the measurement device according to the invention, a transmission device is provided, by means of which the measurement device can be connected to a transmitter, receiver, and evaluation unit arranged outside of the body. The transmission device can be embodied both as an interface, which passes on incoming data, and also as an independent transmitter.

To be able to operate measurement and application methods using electromagnetic radiation, the transmission device has a radiation output for bringing electromagnetic radiation of different frequencies, especially visible light, into the interior of the body cavity. For preparing the radiation, the measurement device can be coupled especially advantageously to the transmitter, receiver, and evaluation unit by means of at least one optical fiber cable.

Another embodiment of the measurement device can be secured against undesired shifting within the body cavity with an additional securing device. For this purpose, the measurement device is provided with at least one additional fastening means, especially a thread holder.

In another embodiment, the measurement device is arranged on a stent cage and thus can be used in combination with this vessel-widening or vessel-stabilizing element, which already requires implantation at the corresponding location. Preferably, the measurement device is integrated into the lattice structure of the stent cage.

In one advantageous improvement of this structure, the measurement device has several sensors, which are connected to magnetic elements and which are arranged in a plane of the stent cage, especially in a uniformly distributed arrangement.

The measurement device according to the invention described above can be used at many locations in the human body for analyzing and administering medicines or, e.g., heat. Here, locations where the device is used include, in addition to veins and arteries, also the stomach, intestines, and the esophagus, the urological and gynecological area, as well as the brain.

### BRIEF DESCRIPTION OF THE DRAWINGS

The invention is explained in more detail below with reference to embodiments in the drawing. Shown in partially schematic view are:

FIGS. 1a-1e a longitudinal section through a body cavity, on whose wall an embodiment of the measurement device is arranged;

FIGS. 2a, 2b a longitudinal section and a cross section through a body cavity, in whose interior there is a stent cage with a measurement device arranged thereon.

### DETAILED DESCRIPTION OF THE PREFERRED EMBODIMENTS

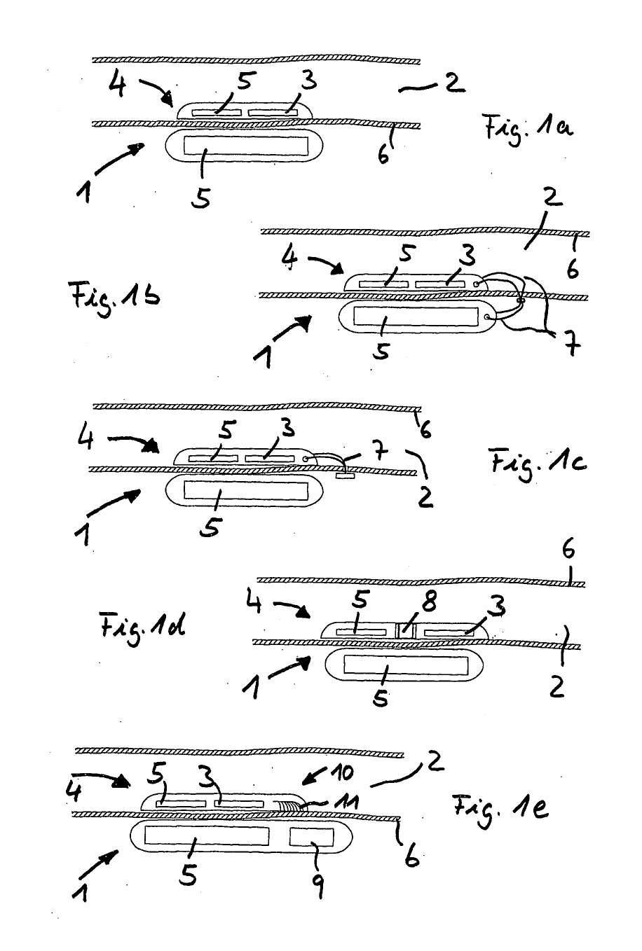

In FIGS. 1a-e, a body cavity 2 in the form of an artery is to be seen, in which a measurement device, designated as a whole with 1, is housed. Here, the measurement device 1 has a sensor 3 for detecting a parameter and is fixed by means of a holder 4 to the vessel wall 6. The holder 4 is formed by a first and a second magnetic element 5, in this embodiment each being a magnet, which are located on both sides of the vessel wall 6. The part of the measurement device 1 located on the inside of the vessel wall 6 comprises a sensor 3 cast in a flexible, flat, boat-like shape and a similarly cast magnet, which is connected to this sensor. Due to its shape, the sensor 3 with its magnetic element 5 as such does not represent a mechanical obstacle for the blood flowing in the artery. On the outside of the vessel wall 6, a holding magnet with approximately the same length is mounted as magnetic element 5, which holds the sensor 3 with the magnetic element on the other side of the vessel wall 6 in its position.

In FIG. 1b, the parts of the measurement device 1 located both on the inside and also on the outside of the vessel wall 6 are each provided with a thread holder 7 as an additional securing device. The threads of the thread holder 7 guided through an eye of the corresponding part are connected to the outside of the vessel by a knot. In contrast, the measurement device 1 illustrated in FIG. 1c has a thread holder only on its part arranged on the inside of the vessel. The thread of this holder is guided in turn through an eye on the part. The end of the thread facing away from the part is provided with an anchor-like piece, which fixes the thread there by its position on the outside of the vessel wall 6.

In FIG. 1d, it will be recognized that for the measurement device 1 illustrated there, there is a storage device 8, which is provided for holding a medicine to be released into the bloodstream, between the magnetic element 5 and the sensor 3. In turn, what follows from FIG. 1e is that for the measurement device 1 an electronic memory unit 9 is arranged as a temporary memory for the data detected by the sensors in the region of the magnetic element 5 on the outside wall of the vessel. In addition, on its part located in the interior of the body cavity 2, the measurement device 1 has a transmission device 10, of which, in FIG. 1e, the interface for coupling visible light through an optical fiber cable 11 is provided.

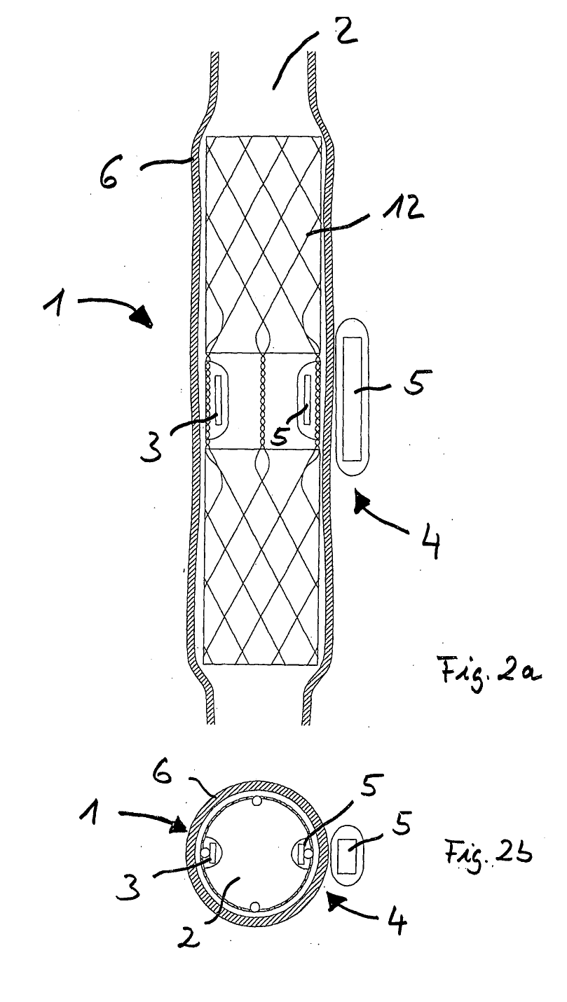

In FIG. 2a, the longitudinal section of a blood vessel is to be seen as the body cavity 2, which is expanded by a stent cage 12 mounted therein. For example, in the center of the stent cage 12, a measurement device 1 with two opposing sensors 3 and magnetic elements 5 is integrated in the lattice structure of the stent cage 12, such that they are mounted on the braces of the lattice structure. On the outside of the vessel wall 6, there is a magnet, which fixes the measurement device and the stent cage within the vessel through its interaction with the magnetic elements 5 of the measurement device 1. FIG. 2b shows an illustration of the same arrangement in a cross section through the body cavity 2.

Consequently, the invention comprises a measurement device 1 for detecting medical parameters in the human body, which can be accommodated in a body cavity 2, especially a blood vessel, with at least one sensor 3 and one holder 4. The measurement device is characterized in that the holder 4 has at least one first and one second magnetic element 5, of which at least one is a magnet and of which one is arranged inside of the body cavity and the other is arranged outside of the body cavity 2 and that the measurement device 1 can be fixed by the holder 4 in the body cavity 2.

1. Measurement device for detecting medical parameters in the human body, which can be accommodated in a body cavity (2), the device comprising at least one sensor (3) and a holder (4), the holder (4) has at least one first and one second magnetic element (5), of which at least one of the magnetic elements is a magnet and of which one of the magnetic elements is arranged inside of the body cavity and the other of the magnetic elements is arranged outside of the body cavity (2), and the measurement device (1) is adapted to be fixed by the holder (4) in the body cavity (2).

2. Measurement device according to claim 1, wherein one of the two magnetic elements (5) is a magnet and the other is a part made from a ferromagnetic material.

3. Measurement device according to claim 1, wherein both of the magnetic elements (5) comprise magnets.

4. Measurement device according to claim 1, wherein the at least one sensor (3) is connected rigidly to the magnetic element (5) arranged inside of the body cavity (2).

5. Measurement device according to claim 1, wherein the magnetic element (5) arranged outside of the body cavity (2) is the magnet.

6. Measurement device according to claim 1, wherein the measurement device is adapted to be moved within the body cavity (2) by rearranging or shifting the magnet.

7. Measurement device according to claim 1, wherein the at least one sensor comprises a plurality of sensors (3), which are provided with the magnetic elements (5) and which can be fixed in the body cavity (2) by at least one magnet.

8. Measurement device according to claim 1, wherein the measurement device is adapted to be inserted into the body cavity (2) via an implantation instrumentor a catheter, or the like.

9. Measurement device according to claim 1, wherein the magnetic element (5) arranged outside of the body cavity (2) is adapted to be applied to a surface of the body or subcutaneously.

10. Measurement device according to claim 1, wherein the measurement device is at least partially sheathed or encased in a flexible, biocompatible material.

11. Measurement device according to claim 10, wherein the measurement device (1), and an electronic component arranged on the device, are provided with an additional coating.

12. Measurement device according to claim 1, wherein the measurement device is provided with a power supply, especially a battery or an accumulator.

13. Measurement device according to claim 1, further comprising an electronic memory unit (9) for temporary storage of data detected by the sensor in a region of the sensor (3) or one of the magnetic elements (5).

14. Measurement device according to claim 13, further comprising an evaluation unit for additional processing of the detected data is provided in a region of the sensor (3) or one of the magnetic elements (5).

15. Measurement device according to claim 1, wherein the at least one sensor comprising a plurality of sensors, the sensors (3) are provided for detecting values of pressure, blood-sugar level, hemoglobin count, oxygen and carbon dioxide partial pressures and content, and/or other selected values of the body cavity and/or a medium located therein.

16. Measurement device according to claim 1, wherein at least one storage device (8) is provided on the measurement device for housing a material to be introduced into the body cavity (2).

17. Measurement device according to claim 16, wherein a dosing element for controlled release of the material is provided on the storage device (8).

18. Measurement device according to claim 17, wherein the measurement device is part of a control loop and the dosing element releases the material as a reaction to a measurement value detected by the sensor.

19. Measurement device according to claim 1, wherein on the measurement device there is a transmission device (10), through which the measurement device (1) can be connected to a transmitter, receiver, and evaluation unit arranged outside of the body using a wireless and/or wired connection.

20. Measurement device according to claim 19, wherein the transmission device (10) has a radiation output for introducing electromagnetic radiation of different frequencies, especially visible light, into an interior of the body cavity (2).

21. Measurement device according to claim 19, wherein the measurement device is coupled to the transmitter, receiver, and evaluation unit via at least one optical fiber cable (11).

22. Measurement device according to claim 1, wherein the measurement device is provided with a thread holder (7).

23. Measurement device according to claim 1, further comprising a stent cage (12) connected to the measurement device.

24. Measurement device according to claim 23, wherein the measurement device is integrated at least partially into a lattice structure of the stent cage (12).

25. Measurement device according to claim 23, wherein the at least one sensor comprises a plurality of sensors, which are connected to the magnetic elements (5) and which are arranged in a plane of the stent cage (12), in a uniformly distributed arrangement.
